(19) United States
(12) Patent Application Publication
(10) Pub. No.: US 2003/0134341 A1
(43) Pub. Date: Jul. 17, 2003

(54) Th1 cell adoptive immunotherapy

(75) Inventor: Micheal L. Gruenberg, Poway, CA (US)

Correspondence Address: HELLER EHRMAN WHITE & MCAULIFFE LLP, 4350 LA JOLLA VILLAGE DRIVE, 7TH FLOOR, SAN DIEGO, CA, 92122-1246, US

(73) Assignee: MedCell Biologics, LLC.

(21) Appl. No.: 09/957,194

(22) Filed: Sep. 19, 2001

(51) Int. Cl. G01N 33/567; C12N 5/08

(52) U.S. Cl. 435/7.21; 435/372

(57) ABSTRACT

A method for consistently producing a pure population of activated, polyclonal, Th1 memory cells for use in adoptive immunotherapy without the use of any exogenous cytokines and without significant subject-to-subject variation is provided. These cells obtain a surface phenotype that enables their trafficking to tumors and other sites of inflammation upon infusion and can be reinfused into the a subject to enhance the cellular immune response and/or switch the predominant immune response from Th2-dominated to a Th1-dominated immune response.

### RELATED APPLICATIONS

This application is related to U.S. application Ser. No. 08/506,668, converted to U.S. provisional application Serial No. 60/044,693, now abandoned; pending U.S. applications Ser. Nos. 08/700,565, 09/127,411, 09/127,142, 09/127,138, 09/127,141, 09/824,906, and International PCT application No. WO 97/05239. The subject matter of each of these applications is incorporated by reference in its entirety.

### FIELD OF THE INVENTION

This invention relates to immunotherapy. In particular, methods for the ex vivo production of autologous T-cells and the resulting T-cells for adoptive immunotherapy are provided.

### BACKGROUND

The immune system is designed to eradicate a large number of pathogens, as well as tumors, with minimal immunopathology. When the immune system becomes defective, however, numerous disease states result. Immunotherapy is an emerging treatment modality that seeks to harness the power of the human immune system to treat disease. Immunotherapy seeks to enhance the cellular immune response in diseases characterized by immunosuppression and suppress the cellular immune response in subjects with diseases characterized by an overactive cellular immune response.

One immunotherapy method for enhancing the cellular immune response in subjects is a type of cell therapy called adoptive immunotherapy. A cell therapy is a drug whose active ingredient is wholly or in part a living cell. Adoptive immunotherapy is a cell therapy that involves the removal of immune cells from a subject, the ex-vivo processing (i.e., activation, purification and/or expansion of the cells) and the subsequent infusion of the resulting cells back into the same subject.

Examples of adoptive immunotherapy methods include methods for producing and using LAK cells (Rosenberg U.S. Pat. No. 4,690,915), TIL cells (Rosenberg U.S. Pat. No. 5,126,132), cytotoxic T-cells (Cai, et al U.S. Pat. No. 6,255,073; Celis, et al. U.S. Pat. No. 5,846,827), expanded tumor draining lymph node cells (Terman U.S. Pat. No. 6,251,385), various preparations of lymphocytes (Bell, et al U.S. Pat. No. 6,194,207; Ochoa, et al. U.S. Pat. No. 5,443,983; Riddell, et al. U.S. Pat. No. 6,040,180; Babbitt, et al. U.S. Pat. No. 5,766,920; Bolton U.S. Pat. No. 6,204,058), CD8+ TIL cells (Figlin et al. (1997) Journal of Urology 158:740), CD4+ T-cells activated with anti-CD3 monoclonal antibody in the presence of IL-2 (Nishimura (1992) J. Immunol. 148:285), T-cells co-activated with anti-CD3 and anti-CD28 in the presence of IL-2 (Garlie et al. (1999) Journal of Immunotherapy 22:336) antigen-specific CD8+ CTL T-cells produced ex-vivo and expanded with anti-CD3 and anti-CD28 monoclonal antibodies (mAb) in the presence of IL-2 (Oelke et al. (2000) Clinical Cancer Research 6:1997), and injection of irradiated autologous tumor cells admixed with Bacille Calmette-Guérin (BCG) to vaccinate subjects followed seven days later by recovery of draining lymph node T-cells which are activated with anti-CD3 mAb followed by expansion in IL-2 (Chang et al. (1997) Journal of Clinical Oncology 15:796).

Adoptive immunotherapy once held great promise as a treatment method, but enthusiasm has since faded. Despite great efforts by academic and commercial laboratories, none of these prior art adoptive immunotherapy methods have been approved by the Food and Drug Administration (FDA) as a therapy to treat human disease. There have been numerous obstacles in obtaining FDA approval for this type of treatment regimen. The most significant have been the infrequent and sporadic efficacy and high toxicity associated with these treatments. The reasons for the infrequent and sporadic efficacy of these treatments is not clearly understood, but may be related to the types, purity and dosages of cells used in the protocols, as well as subject-to-subject variations. The high toxicity is associated with the requirement that immune cells that have been processed ex-vivo be infused concomitantly with the highly toxic growth factor, interleukin-2 (IL-2), in order to maintain their viability and function.

Prior art adoptive immunotherapy methods have focused on the differentiation and expansion of effector cells (e.g, LAK, NK and CTL). A new immunological paradigm has emerged that has taught the importance of regulatory cells, such as Th1 and Th2 cells, in the immune response. For the most part, current adoptive immunotherapy methods do not take advantage of this knowledge.

Functionally distinct regulatory cells, called Th1 and Th2, are found in both mice and in humans (Mosmann et al (1989) Advances in Immunology 46:111; Romagnani et al (1991) Immunology Today 12:256). The functional division of CD4+ lymphocytes into Th1 and Th2 subsets is based upon their cytokine profile. Th1 cells produce gamma interferon (IFN-γ) and interleukin-2 (IL-2), but not IL-4. Th2 cells produce IL-4, but not IFN-γ (Mosmann et al (1989) Advances in Immunology 46:111; Mosmann et al. (1989) Annual Review of Immunology 7:145; Mosmann et al. (1986) Journal of Immunology 136:2348; Fiorentino et al. (1989) Journal of Experimental Medicine 170:2081). Cytokines produced by these two subsets are mutually inhibitory and establish a reciprocal cross regulation. Th1 cells inhibit the proliferation of Th2 cells and Th2 cells inhibit Th1 cell cytokine production (Fiorentino et al. (1989) Journal of Experimental Medicine 170:2081). This cross regulation results in a polarized Th1 or Th2 immune response to pathogens that can determine either host resistance or susceptibility to infection. For example, a Th1 response in protozoan, viral or fungal infection is associated with resistance, while a Th2 response to these pathogens is associated with disease (Sher et al. (1992) Immunological Reviews 127:183; Scott et al. (1991) Immunology Today 12:346).

These observations have led to a new immunological paradigm. It is now widely believed that the homeostasis of the immune system is regulated by the balance of cytokines produced by Th1 and Th2 lymphocyte subsets (Tanaka et al. (1998) Rinsho Byori Japanese Journal of Clinical Pathology 46:1247), whereas imbalances in Th1/Th2 cytokines correlates with disease (Shurin et al. (1999) Seminars in Immunopathology 21:339).

Copending U.S. application Ser. No. 08/506,668, converted to U.S. provisional application Serial No. 60/044,693, now abandoned; pending U.S. applications Ser. Nos. 08/700,565, 09/127,411, 09/127,142, 09/127,138, 09/127,141, 09/824,906, and International application No. WO 97/05239 provide methods for ex-vivo T-cell expansion from subjects without the use of exogenous IL-2 and methods for producing compositions of T-cells, including Th1 cells, that are predominately Th1 (greater than 50%) for the treatment of a variety of diseases. The methods described therein require the use of exogenous cytokines to cause the differentiation of Th1 cells. Further improvement of the methods described therein to consistently produce more homogenous populations of Th1 cells without the use of exogenous cytokines and with a phenotype that would enable their trafficking to tumors and other sites of inflammation is desirable.

There is a need to develop new methods for adoptive immunotherapy that permit consistent production of cell products that do not vary subject-to-subject and do not require the use of exogenous cytokines or the concomitant infusion of toxic growth factors. There is also a need to develop new cell compositions that improve the efficacy associated with this therapeutic approach.

Accordingly, it is an object herein to provide such methods. It is an object herein to provide an immunotherapeutic technology that employs natural immunoregulatory mechanisms to stimulate an immune response.

### SUMMARY OF THE INVENTION

Methods for consistently producing a population of highly pure, activated, polyclonal memory Th1 cells from a subject blood sample in the absence of any exogenous growth or differentiation factors (such as IL-2 or IFN-γ) for use in adoptive immunotherapy are provided. The cells produced by the methods can be used to enhance the cellular immune response or to switch a Th2-dominated immune response to a Th1-dominated immune response in subjects. These cells have therapeutic application in subjects suffering from a variety of diseases, including cancer, infectious diseases, aging, allergic and other inflammatory diseases and diseases characterized by overactive humoral immunity (such as in systemic lupus erythematosus).

The methods provided herein include the steps of: (i) collecting source material from a subject; (ii) purifying T-cells from the source material; (iii) activating frequently (such as every 2-3 days) and repeatedly (a minimum of 3 times for the exemplified embodiment) the purified T-cells; and optionally (iv) reinfusing the resulting cells into the same subject or an allogeneic recipient.

The purification step minimizes the subject-to-subject variability of the cells resulting from the process. The frequent and repeated activation step causes the differentiation and expansion of a highly pure population of activated, polyclonal, memory Th1 cells. The methods do not require the addition of any exogenous growth or differentiation factors.

It is shown herein that frequent restimulation of T-cells or T-cell subsets with, for example, immobilized anti-CD3 and anti-CD28 mAb, causes them to proliferate and differentiate into a highly pure population of activated memory Th1 cells useful for adoptive immunotherapy of diseases characterized by either a lack of cellular immunity or an excess of humoral immunity. The frequency of the restimulation must be every 2-3 days and the restimulation must be repeated at least 3 and typically 4 times in order to obtain a pure population of activated Th1 memory cells. Activation with these antibodies greater than 5 times, however, results in diminishing cytokine production and increased activation-induced cell death.

The source material contains mononuclear cells collected from a blood sample, such as by leukapheresis. According to a method provided herein, a population of CD3+ T-cells is first purified from the source material. In an exemplary embodiment of the method herein, the source material is first purified to obtain a starting population of CD4+ T-cells. The CD4+ cells are purified, for example, by positive selection techniques. In another exemplary embodiment, the purified CD4+ T-cells are purged of CD45RO+ memory cells resulting in a starting population of CD4+, CD45RA+ naive T-cells (pTh cells).

The starting population of T-cells (either CD3+ or CD4+ or CD4, CD45RA+) are next frequently and repeatedly activated. In one embodiment, the cells are activated by simultaneous contact with a first agent that stimulates the TCR/CD3 complex of the T-cells and a second agent which stimulates the CD28 receptor complex. In another exemplary embodiment, the activation is accomplished by co-incubating the starting population of T-cells with immunomagnetic beads conjugated with anti-CD3 and anti-CD28 monoclonal antibodies.

The frequent and repeated re-activation causes the cells to expand and differentiate. In order to cause Th1 memory differentiation, the T-cells must be reactivated at least 3 times, typically 4 times, every 2-4 days, generally every 3 days. In an exemplary embodiment, the T-cells are reactivated 4 times with anti-CD3/anti-CD28-conjugated immunomagnetic beads every 3 days.

The frequent and repeated activation results in cells that expand in excess of 100-fold in the absence of exogenous growth factors, such as IL-2. In order to consistently expand cells from subjects with cancer without the use of exogenous cytokines, the source material must be first purged of platelets. The platelets from cancer subjects are a source of TGF-beta, which inhibits the expansion of T-cells. In other embodiments, the source material is purged of monocytes prior to purification of CD4+ T-cells by positive selection.

Compositions containing the cells resulting from the method are provided. These cells can be used, for example, to treat cancers, infectious diseases, allergic diseases and suppress the humoral immune response in diseases characterized by overactive humoral immunity. The cells resulting from the method have a unique phenotype: CD3+, CD45RO+, CD25+, CD40L+, CD62L−, CD44+. The cells internally stain positive for IFN-γ and do not produce IL-4. The cells produce proinflammatory Th1 cytokines, including IFN-γ, TNF-alpha and IL-2. Cells of this phenotype, referred to herein as activated, polyclonal memory Th1 cells, have the ability to leave the vasculature upon reinfusion and enter cancerous lesions and other sites of inflammation.

The capability to deliver proinflammatory Th1 cytokines within tumors and sites of inflammation can shift a resident immune response from a Th2-dominated response to a Th1-dominated immune response. Th1 cytokines also have a proven ability to act as a general booster of the cellular immune response. This is a unique mechanism of action for cells used in adoptive immunotherapy and will be beneficial to subjects suffering from various forms of cancer, infectious diseases and allergic diseases, and other diseases characterized by either suppressed cellular immunity or enhanced humoral immunity.

The method consistently produces high purity activated, polyclonal memory Th1 cells without any exogenous cytokines, such as IL-2, and without significant subject-to-subject variation. This permits the cells to be infused without the concomitant infusion of IL-2. The consistent production of a pure cell product combined with the unique mechanism of action and lack of toxic cytokines is an improvement over prior art adoptive immunotherapy methods and should result in an improved therapeutic index.

The method also can enhance the activated, Th1 memory cell component of a population of cells that result when the source cells are CD3+, CD4+, CD45RO+ memory cells. Such enhancement occurs even in memory cells derived from a subject with a Th2-dominated disease, such as a cancer subject. The method herein, which relies on repeated and frequent activation, causes the endogenous production of large amounts of IFN-γ, which inhibits Th2 cytokine production. The method also preferentially expands Th1 cells. Thus, even a starting Th2 cell-enriched population, such as CD4+, CD45RO+ memory cells from cancer subjects, when treated in accord with the methods herein, produces a population that is enhanced in Th1 memory cells.

Also provided herein vaccines that are a combination of the cells produced herein and an immunizing antigen, and methods of vaccinating by co-infusing, either sequentially or simultaneously, the cells produced herein and an immunizing antigen. Immunizing antigens include but are not limited to, tumor-associated antigens, viral antigens bacterial antigens and other antigens against which an immunoprotective or disease-ameliorative response is desired.

Methods of treatment of diseases characterized by suppression of the cellular immune response or by over-expression of the humoral immune response are provided. Such diseases include, but are not limited to, cancer, infectious diseases, autoimmune, inflammatory resposne, allergic diseases and aging. The cells produced by each of the methods provided herein are adiminstered to the donor of the cells or to an allogenic recipient. A sufficient number of cells are administered to ameliorate the symptoms of the disease. Typically at least about 10 ⁸-10¹¹ cells, generally at least about 10⁹ cells are administered either as a single dosage or in several dosages.

### DETAILED DESCRIPTION

A. Definitions

Unless defined otherwise, all technical and scientific terms used herein have the same meaning as is commonly understood by one of ordinary skill in the art to which this invention belongs. All patents, applications, published applications and other publications referred to throughout the disclosure herein are incorporated by reference in their entirety.

As used herein, cell therapy is a method of treatment involving the administration of live cells. Adoptive immunotherapy is a treatment process involving removal of cells from a subject, the processing of the cells in some manner ex-vivo and the infusion of the processed cells into the same subject as a therapy.

As used herein, source material is the population of cells that are collected from a subject for further processing into an adoptive immunotherapy. Source material generally is mononuclear cells collected, for example, by leukapheresis.

As used herein, immune cells are the subset of blood cells known as white blood cells, which include mononuclear cells such as lymphocytes, monocytes, macrophages and granulocytes.

As used herein, T-cells are lymphocytes that express the CD3 antigen.

As used herein, helper cells are CD4+ lymphocytes.

As used herein, regulatory cells are a subset of T-cells, most commonly CD4+ T-cells, that are capable of enhancing or suppressing an immune response. Regulatory immune cells regulate an immune response primarily by virtue of their cytokine secretion profile. Some regulatory immune cells can also act to enhance or suppress an immune response by virtue of antigens expressed on their cell surface and mediate their effects through cell-to-cell contact. Th1 and Th2 cells are examples of regulatory cells.

As used herein, effector cells are immune cells that primarily act to eliminate tumors or pathogens through direct interaction, such as phagocytosis, perforin and/or granulozyme secretion, induction of apoptosis, etc. Effector cells generally require the support of regulatory cells to function and are the mediators of delayed type hypersensitivity reactions and cytotoxic functions. Examples of effector cells are B lymphocytes, macrophages, cytotoxic lymphocytes, LAK cells, NK cells and neutrophils.

As used herein, an activated cell is a T-cell that expresses CD25. Cells that express the IL-2 receptor (CD25) are referred to herein as “activated”. A pure or highly pure population of activated cells typically express greater than 85% positive for CD25.

As used herein, T-cells that produce IFN-γ, and not IL-4 upon stimulation are referred to as Th1 cells. Cells that produce IL-4, and not IFN-γ, are referred to as Th2 cells. A method for identifying Th1 cells in a population of cells is to stain the cells internally for IFN-γ. Th2 cells are commonly identified by internal staining for IL-4. Normal (i.e., subjects not exhibiting overt disease) individuals generally only about 12-16% of the CD4+ cells stain positive for internal IFN-γ after activation; less than 1% stain positive for IFN-γ prior to activation. It is rare for a T-cell population to stain greater than 35% IFN-γ positive. The cells resulting from the method stain greater than 70% positive and often greater than 90% positive for IFN-γ.

As used herein, a pure or highly pure population of Th1 cells is a population that stains greater than 70% positive for internal interferon-γ and does not produce greater than about 26 pg/ml/10⁶ cells of IL-4 in a 24 hour period. In most instances, they do not produce greater than about 6 pg/ml/10⁶ cells of IL-4 in a 24 hour period.

As used herein, a memory cell is a T-cell that expresses CD45RO and not CD45RA. A pure or highly pure population of memory cells expresses greater than 70%, generally greater than 80%, and even greater than 90% or 95% positive for CD45RO.

As used herein, a cell that has the ability to traffic to a tumor or other site of inflammation upon infusion, is a T-cell with an activated (CD25+) memory (CD45RO+) phenotype that expresses adhesion molecules, such as CD44 and does not expresses CD62L. A pure or highly pure population of memory cells with the ability to traffic to a tumor or other site of inflammation upon infusion is greater than 70%, generally greater than 90% or 95% positive for CD44, and less than about 25%, including less than 5%, positive for CD62L.

As used herein, activating proteins are molecules that when contacted with a T-cell population cause the cells to proliferate. Reference to activating proteins thus encompasses the combination of proteins that provide the requisite signals, which include an initial priming signal and a second co-stimulatory signal. The first signal requires a single agent, such as anti-CD3 monoclonal antibody (mAb), anti-CD2 mAb, anti-TCR mAb, PHA, PMA, and other such signals. The second signal requires one or more agents, such as anti-CD28 mAb, anti-CD40L mAb, cytokines, feeder cells or other such signals. Thus activating proteins include combinations of molecules including, but are not limited to: cell surface protein specific mAbs, fusion proteins containing ligands for a cell surface protein, or any molecule that specifically interacts with a cell surface receptor on a T-cell and directly or indirectly causes that cell to proliferate.

As used herein, a mitogenic mAb is an activating protein that is a monoclonal antibody specific for a T-cell surface expressed protein that when contacted with a cell directly or indirectly provides one of the at least two requisite signals for T-cell mitogenesis. Suitable mitogenic mAbs induce T-cell doubling times of 24 h to 48 h.

As used herein, a cytokine is a factor produced from a cell that has biological activity. A lymphokine is a cytokine produced by lymphocytes. Interleukins and interferons are examples of lymphokines.

As used herein, exogenous cytokines, refer to cytokines that are added to a sample or cell preparation. They do not include cytokines produced by the cells in a sampel or cell preparation in vitro, in vivo or ex vivo. Hence preparing cells in the absence of exogenous cytokines, refers to preparation without adding additional cytokines to those produced by the cells.

As used herein, a composition containing a clinically relevant number or population of immune cells is a composition that contains at least 10⁹, typically greater than 10⁹, at least 10¹⁰ cells, and generally more than 10¹⁰ cells. The number of cells will depend upon the ultimate use for which the composition is intended as will the type of cell. For example, if Th1 cells that are specific for a particular antigen are desired, then the population will contain greater than 70%, generally greater than 80%, 85% and 90-95% of such cells. For uses provided herein, the cells are generally in a volume of a liter or less, can be 500 mls or less, even 250 mls or 100 mls or less. Hence the density of the desired cells is typically be greater than 10⁶ cells/ml and generally is greater than 10⁷ cells/ml. The clinically relevant number of immune cells can be apportioned into multiple infusions that cumulatively equal or exceed 10⁹, 10¹⁰ or 10¹¹ cells.

As used herein, a clinically relevant number of activated polyclonal Th1 memory cells is a composition containing a clinically relevant number or population of immune cells where a substantial portion, greater than at least about 70%, typically more than 80%, 90%, and 95%, of the immune cells are activated polyclonal Th1 memory cells.

As used herein, polyclonal means cells derived from two or more cells of different ancestry or genetic constitution. A polyclonal T-cell population is a population of T-cells that express a mixture of T cell receptor genes with no one T cell receptor gene dominating the population of cells.

As used herein, predominant means greater than about 50%.

As used herein, highly pure means greater than about 70%, generally greater than 75% and can be as pure as 85%, 90% or 95% or higher in purity. A highly pure population of Th1 cells, as used herein, is typically a population of greater than 95% CD3+, CD4+ T-cells that stain greater than about 70% positive for internal IFN-γ and do not produce detectable amounts of IL-4 when assayed by ELISA (i.e., less than 26 pg/ml/10⁶ cells). Internal staining for IL-4 is generally below 10% and most often below 5%. Occasionally higher numbers are observed. This is often an artifact of the detection technique, as cells that die by apoptosis will stain positive for internal IL-4. Measurement of secretion into supernatants controls for this artifact. The amount of IFN-γ detected by ELISA is generally in excess of 1 ng/ml/10⁶ cells and in the range of 1 ng/ml to 26 ng/ml per 10⁶ cells, but can be greater than 26 ng/ml per 10⁶ cells.

As used herein, a combination refers to two component items, such as compositions or mixtures, that are intended for use either together or sequentially. The combination may be provided as a mixture of the components or as separate components packaged or provided together, such as in a kit.

As used herein, effector cells are mononuclear cells that have the ability to directly eliminate pathogens or tumor cells. Such cells include, but are not limited to, LAK cells, MAK cells and other mononuclear phagocytes, TILs, CTLs and antibody-producing B cells and other such cells.

As used herein, immune balance refers to the normal ratios, and absolute numbers, of various immune cells and their cytokines that are associated with a disease free state. Restoration of immune balance refers to restoration to a condition in which treatment of the disease or disorder is effected whereby the ratios of regulatory immune cell types or their cytokines and numbers or amounts thereof are within normal range or close enough thereto so that symptoms of the treated disease or disorder are ameliorated. The amount of cells to administer can be determined empirically, or, such as by administering aliquots of cells to a subject until the symptoms of the disease or disorder are reduced or eliminated. Generally a first dosage will be at least 10⁹-10¹⁰ cells. In addition, the dosage will vary depending upon treatment sought. As intended herein, about 10⁹ is from about 5×10⁸ up to about 5×10⁹; similarly about 10¹⁰ is from about 5×10⁹ up to about 5×10¹⁰, and so on for each order of magnitude. Dosages refer to the amounts administered in one or in several infusions.

As used herein, therapeutically effective refers to an amount of cells that is sufficient to ameliorate, or in some manner reduce the symptoms associated with a disease. When used with reference to a method, the method is sufficiently effective to ameliorate, or in some manner reduce the symptoms associated with a disease.

As used herein, a subject is a mammal, typically a human, including patients.

As used herein, mononuclear or lymphoid cells (the terms are used interchangeably) include lymphocytes, macrophages, and monocytes that are derived from any tissue or body fluid in which such cells are present. In general lymphoid cells are removed from an individual who is to be treated. The lymphoid cells may be derived from a tumor, peripheral blood, or other tissues, such as the lymph nodes and spleen that contain or produce lymphoid cells.

As used herein, a therapeutically effective number is a clinically relevant number of immune cells that is at least sufficient to achieve a desired therapeutic effect, when such cells are used in a particular method. Typically such number is at least 10⁹, and generally 10¹⁰ or more. The precise number will depend upon the cell type and also the intended target or result and can be determined empirically.

As used herein, tissue culture medium includes any culture medium that is suitable for the growth of mammalian cells ex vivo. Examples of such medium include, but are not limited to X-VIVO-15 (Biowhittaker) AIM-V, RPMI 1640, and Iscove's medium (GIBCO, Grand Island, N.Y.). The medium may be supplemented with additional ingredients including serum, serum proteins, growth suppressing, and growth promoting substances, such mitogenic monoclonal antibodies and selective agents for selecting genetically engineered or modified cells.

As used herein, a disease characterized by a lack of Th1 cytokine activity refers to a state, disease or condition where the algebraic sum of cytokines in a specific microenvironment in the body or in a lesion(s) or systemically is less than the amount of Th1 cytokines present normally found in such microenvironment or systemically (i.e., in the subject or another such subject prior to onset of such state, disease or condition). The cytokines to assess include IFN-γ, IL-2, and TNFα. The precise amounts and cytokines to assess depend upon the particular state, disease or condition. Thus, the diseases for which the cells have therapeutic application include, but are not limited to, cancer, infectious diseases, allergic diseases and diseases characterized by overactive humoral immunity (such as in systemic lupus erythematosus).

As used herein, diseases characterized by a Th2-dominated immune response are characterized by either a suppressed cellular immune response or excessive humoral response.

As used herein, a disease characterized by a an excess of Th2 cytokine activity refers to a state, disease or condition where the algebraic sum of cytokines in a specific microenvironment in the body or in a lesion(s) or systemically is predominantly of the Th2 type, dominated by IL-4 and/or IL-10 and/or TGF-β. Diseases, states or conditions that exhibit enhanced Th2 responses include infectious diseases such as, but are not limited to, chronic hepatitis C virus infection, leprosy toxoplasmosis infection and AIDS. Imbalance in favor of Th2 cells also occurs in asthma and lupus and other diseases that exhibit suppressed cellular immunity.

Thus, the cells produced by the methods herein, which are predominantly Th1 cells, are used to treat diseases characterized by an excess of Th2 cytokine activity or lack of Th1 cytokine activity. Hence methods for treatments of such diseases are provided. The methods and cells enhance the cellular immune response or effect a switch from a Th2-dominated immune response to a Th1-dominated immune response in subjects.

As used herein, treatment means any manner in which the symptoms of a condition, disorder or disease are ameliorated or otherwise beneficially altered. Treatment also encompasses any pharmaceutical use of the compositions herein.

As used herein, a vaccine is a composition that provides protection against a viral infection, cancer or other disorder or treatment for a viral infection, cancer or other disorder. Protection against a viral infection, cancer or other disorder will either completely prevent infection or the tumor or other disorder or will reduce the severity or duration of infection, tumor or other disorder if subsequently infected or afflicted with the disorder. Treatment will cause an amelioration in one or more symptoms or a decrease in severity or duration. For purposes herein, a vaccine results from co-infusion (either sequentially or simultaneously) of an antigen and a composition of cells produced by the methods herein. As used herein, amelioration of the symptoms of a particular disorder by administration of a particular composition refers to any lessening, whether permanent or temporary, lasting or transient that can be attributed to or associated with administration of the composition.

As used herein, substantially pure means sufficiently homogeneous to appear free of readily detectable impurities as determined by standard methods of analysis, such as flow cytometry, used by those of skill in the art to assess such purity, or sufficiently pure such that further purification would not detectably alter the physical and chemical properties, such as biological activities, of the substance. Methods for purification of the immune cells to produce substantially pure populations are known to those of skill in the art. A substantially pure cell population, may, however, be a mixture of subtypes; purity refers to the activity profile of the population. In such instances, further purification might increase the specific activity of the cell population.

As used herein, biological activity refers to the in vivo activities of immune cells or physiological responses that result upon in vivo administration of a cell, composition or other mixture. Biological activity, thus, encompasses therapeutic effects and pharmaceutical activity of such cells, compositions and mixtures.

Although any similar or equivalent methods and materials can be employed in the practice of the methods and cells provided herein, exemplary embodiments are described.

B. Problems with Prior Art Methods and Solutions Provided Herein

The methods provided herein overcome the problems that have hindered prior art adoptive immunotherapy protocols. The infrequent and sporadic efficacy of prior art adoptive cell immunotherapy protocols is a major reason for the failure of these therapies. One reason for the infrequent and sporadic efficacy is associated with the variations in the source material collected from subjects. Subjects present with a wide variety of hematological profiles. These variations are especially apparent in cancer subjects that have been previously treated with cytotoxic chemotherapy drugs. Due to the fact that the source material collected from each individual subject is different, it is not unexpected that the prior art adoptive immunotherapy protocols that utilized unpurified source material resulted in a final cell population that varied subject to subject. These variations can explain the sporadic efficacy observed in these prior art methods.

Another reason for the infrequent and sporadic efficacy of prior art adoptive cell immunotherapy is due to the inherent immunosuppression of subjects with cancer and some infectious diseases. In many cases the mechanisms that originally suppressed the host immune response from eradicating the tumor or the pathogen are too powerful and well established for prior art adoptive immunotherapy methods to overcome. The cells that result from these methods are capable of producing significant quantities of immunostimulatory cytokines even in the presence of immunosuppressive cytokines. Further, the Th1 cytokines produced by the cells resulting from the methods provided herein, due to their cross regulatory action, can down regulate Th2-mediated suppressive action in these subjects.

Th1 Cells for Adoptive Immunotherapy

Th1 cells promote the cytotoxic and inflammatory reactions, such as delayed type hypersensitivity (DTH) mediated through effector cells such as NK cells, cytotoxic T lymphocytes (CTL) and macrophages. Th2 cells suppress the cellular immune response and promote antibody (Ab) production, isotype switching and eosinophilic inflammation (Mosmann et al. (1989) Annual Review of Immunology 7:145; Yamamura (1992) Science 255:12; Yamamura et al. (1991) Science 254:277 (published erratum appears in Science Jan. 3, 1992;255(5040):12; and Cher et al. (1987) Journal of Immunology 138:3688). Cancer subjects and tumor-bearing animals have been shown to exhibit suppressed cellular immune responses as evidenced by decreased DTH, CTL function and NK activity (Broder (1978) Journal of Medicine 299:1335), not because of lack of effector cells but rather due to a lack of Th1 regulatory cells. The effector cells are present in these subjects in sufficient quantities, but do not function because of a lack of Th1 regulatory cell help. Prior art adoptive immunotherapy sought to enhance the number of effector cells (NK, cytotoxic T-cells and macrophages). Without Th1 regulatory cell support the infused effector cells are as impotent as the resident effector cells, explaining the poor efficacy of prior art methods.

Enhanced Th2 responses, creating an immunosuppressive state, are present in infectious diseases such as chronic hepatitis C virus infection (Fan et al. (1998) Mediators of Inflammation 7:295), leprosy (Yamamura (1992) Science 255:12), toxoplasmosis infection (Sher et al. (1992) Immunological Reviews 127:183) and AIDS (Clerici et al. (1993) Immunology Today 14:107). Imbalance in favor of Th2 cells also occurs in asthma (Robinson et al. (1992) New England Journal of Medicine 326:298) and lupus (Funauchi et al. (1998) Scandinavian Journal of Rheumatology 27:219).

Excess production of Th2 cytokines and/or depressed production of Th1 cytokines resulting in a Th1/Th2 cytokine imbalance has also been reported in virtually all types of cancer tested, including renal cell carcinoma (RCC) (Onishi et al. (1999) Bju International 83:488; Elsässer-Beile et al. (1998) Tumour Biology 19:470; Nakagomi et al. (1995) International Journal of Cancer 63:366; Schoof et al. (1993) Cellular Immunology 150:114; Wang et al. (1995) International Journal of Cancer 61:780), melanoma (Chen et al. (1994) International Journal of Cancer 56:755; Krüger-Krasagakes et al. (1994) Journal of Cancer 70:1182; Fortis et al. (1996) Cancer Letters 104:1), prostate cancer (Hrouda et al. (1998) British Journal of Urology 82:568), digestive cancer (Tabata et al. (1999) American Journal of Surgery 177:203), colon cancer (Berghella et al. (1998) Cancer Immunology, Immunotherapy 45:241), colorectal cancer (Pellegrini et al. (1996) Cancer Immunology, Immunotherapy 42:1), pancreatic and gastric adenocarcinoma (Fortis et al. (1996) Cancer Letters 104:1; Bellone et al. (1999) American Journal of Pathology 155:537), head and neck cancer (Prasad et al. (1998) Journal of the American College of Nutrition 17:409), non-small cell lung cancer (Asselin-Paturel et al. (1998) Journal of Cancer 77:7; Huang et al. (1996) Journal of Immunology 157:5512), lung cancer (Chen et al. (1997) Chest 112:960; Ito et al. (1999) Cancer 85:2359), bronchogenic carcinoma (Smith et al. (1994) American Journal of Pathology 145:18), gynecological tumors (Punnonen et al. (1998) Cancer 83:788; al-Saleh et al. (1998) Journal of Pathology 184:283; Jacobs et al. (1998) Clinical and Experimental Immunology 111:219), breast cancer (Rosen et al. (1998) Cancer Letters 127:129; Goedegebuure et al. (1997) Cellular Immunology 175:150), ovarian cancer (Goedegebuure et al. (1997) Cellular Immunology 175:150), B cell chronic lymphocytic leukemia (de Totero et al. (1999) British Journal of Haematology 104:589), cutaneous T-cell lymphoma (Hirshberg et al. (1999) American Journal of Hematology 60:143; Di Renzo et al. (1997) Immunology 92:99), gastric lymphoma (Hauer et al. (1997) Journal of Clinical Pathology 50:957), T-cell leukemia and the Sezary syndrome (Saed et al. (1994) Journal of Investigative Dermatology 103:29; Tendler et al. (1994) Cancer Research 54:4430), Hodgkin's disease (Serrano et al. (1997) Haematologica 82:542; Clerici et al. (1994) European Journal of Cancer 30A:1464; Damle et al. (1991) Cancer Immunology, Immunotherapy 34:205), thymoma (Fujisao (1998) British Journal of Haematology 103:308), glioma (Huettner et al. (1995) American Journal of Pathology 146:317; Roussel et al. (1996) Clinical and Experimental Immunology 105:344) glioblastoma (Ashkenazi et al. (1997) Neuroimmunomodulation 4:49), basal and squamous cell carcinoma (Kim et al. (1995) Journal of Immunology 155:2240; Yamamura et al. (1993) Journal of Clinical Investigation 91:1005).

A Th1 immune response to a tumor is protective, while a Th2 response permits tumors to implant and progress. For example, in murine models of B cell leukemia/lymphoma and melanoma the animals susceptible to tumor challenge developed a Th2 immune response, while animals that developed a Th1 immune response were protected (Lee et al. (1997) Blood 90:1611). IL-4 released by tumor-associated Th2 cells in mice receiving B16 melanoma cells strongly enhances the extent of pulmonary metastases (Kobayashi et al. (1998) Journal of Immunology 160:5869). Conversely, Th1 cytokine expression has been associated with spontaneously regressing melanoma in humans (Lowes et al. (1997) Journal of Investigative Dermatology 108:914). Similarly, Th2 cytokine dominance is associated with hematopoietic suppression, while Th1 dominance is associated with hematopoietic improvement after thymectomy in subjects with thymoma (Fujisao (1998) British Journal of Haematology 103:308). Subjects with digestive cancers have been shown to have a significant increase in the proportion of Th2-producing cells compared to healthy controls. The proportion of these cells were significantly reduced one month after surgical excision of the tumor (Tabata et al. (1999) American Journal of Surgery 177:203). Similarly, a Th1-dominated immune response was found in tumor tissue of operable subjects with lung cancer and a Th1 to Th2 shift occurred with tumor progression (Ito et al. (1999) Cancer 85:2359). In murine renal cell carcinoma (RCC) and colon adenocarcinoma a gradual loss of Th1 cells and an increase in Th2 cytokines was shown to occur as tumor growth progressed (Ghosh et al. (1995) Journal of the National Cancer Institute 87:1478). Mice bearing primary MC tumors had significantly diminished T-cell and NK-cell functions and impaired capacity to produce Th1 cytokines (Horiguchi et al. (1999) Cancer Research 59:2950). In subjects with RCC, an increase in Th2 cytokines was observed that correlated with the stage and grade of the malignancy (Onishi et al. (1999) Bju International 83:488). Similar findings have been reported in subjects with other types of advanced cancers (Sato et al. (1998) Anticancer Research 18:3951).

Immunosuppression in Cancer Subjects

The immunosuppressive environment in a cancer subject is created in large part by the tumor cells. Tumors appear to produce or create a Th2-biased environment (immunosuppressive environment), which protects the tumor against an immune attack. Tumor cells create a cytokine milieu capable of suppressing an anti-tumor immune response by down-regulating the function of Th1 cells.

Tumor cells are known produce a variety of Th2 cytokines (Chen et al. (1994) International Journal of Cancer 56:755; Asselin-Paturel et al. (1998) Journal of Cancer 77:7; Smith et al. (1994) American Journal of Pathology 145:18; Vowels et al. (1994) Journal of Investigative Dermatology 103:669; Nitta et al. (1994) Brain Research 649:122). Tumor infiltrating cells also produce Th2 cytokines (Roussel et al. (1996) Clinical and Experimental Immunology 105:344). Freshly isolated RCC cells produce IL-10 (Nakagomi et al. (1995) International Journal of Cancer 63:366; Wang et al. (1995) International Journal of Cancer 61:780), a Th2 cytokine. IL-10 is a potent inhibitor of tumor cytotoxicity (Nabioullin et al. (1994)Journal of Leukocyte Biology 55:437) and reduces the proliferation and IFN-γ_production (a Th1 cytokine) of peripheral blood T-cells and T-cell clones (Taga et al. (1993) Journal of Immunology 150:4754; de Waal Malefyt et al. (1993) Journal of Immunology 150:4754). RCC supernatants increase the production of IL-10 from macrophages (Ménétrier-Caux et al. (1999) British Journal of Cancer 79:119). The Th2 cytokines, IL-10 and IL-4, are also produced by RCC TIL (Schoof et al. (1993) Cellular Immunology 150:114; Wang et al. (1995) International Journal of Cancer 61:780; Maeurer et al. (1995) Cancer Immunology, Immunotherapy 41:111). IL-10 serum levels are increased in sera of subjects with solid tumors and correlates with poor responsiveness and decreased survival (De Vita et al. (2000) Oncology Reports 7:357). Increased serum concentrations of IL-10 can be a predictor of unfavorable outcome in RCC (Elsässer-Beile et al. (1999) Cancer Immunology, Immunotherapy 48:204). RCC cells also produce other immunosuppressive cytokines, such as IL-6, IL-8 and TGF-β.

IL-8 suppresses the toxicity and can significantly ablate the anti-tumor effect of IL-2 (Heniford et al. (1994) Journal of Surgical Research 56:82). TGF-β inhibits IFN-γ-induced class II MHC expression (Banu et al.(1999) Kidney International 56:985), preferentially induces APC to secrete IL-10, and concomitantly suppresses the production of the Th1-inducing cytokine, IL-12 (D'Orazio et al. (1998) Journal of Immunology 160:2089) and suppresses antigen-specific activation and cytokine secretion by memory Th1 cells (Lúdvíksson et al. (2000) European Journal of Immunology 30:2101). The cytokines produced by RCC have also been shown to modulate T lymphocyte blast formation (Knoefel et al. (1997) Journal of Interferon and Cytokine Research 17:95). These data show that tumor cells create a cytokine milieu capable of suppressing an anti-tumor immune response by down-regulating the function of Th1 cells.

Although human tumors are often infiltrated by a variety of inflammatory cells, these cells are ineffective. Subjects with RCC often have infiltrating lymphocytes capable of recognizing and responding to autologous tumor (Finke et al. (1994) Journal of Immunotherapy with Emphasis on Tumor Immunology 15:91; Finke et al. (1992) Journal of Immunotherapy 11:1). Tumor growth occurs despite the presence of these cells. The local production of Th2 cytokines by the tumor cells explains the ineffectiveness of these infiltrating cells.

These observations explain why attempts at adoptive immunotherapy using immune effector cells such as LAK, TIL and CTL have resulted in limited efficacy. The failure of an effective antitumor immune response appears to be due primarily to a deficiency of Th1 cells, rather than an absence of effector cells capable of recognizing tumors. Effector cells would not be expected to function in hosts with Th2-dominated immunity. These effector cells require a Th1-dominated environment to function.

Accordingly, it is not desirable to infuse effector cells into subjects with Th2-dominated immunity. Effector cells cannot function in the immunosuppressive environment of hosts with cancer and infectious diseases.

Restoring Th1/Th2 Balance is Therapeutic

In order to overcome the immunosuppression in hosts with cancer and infectious diseases, it is desirable to correct the Th1/Th2 imbalance. Adoptive transfer studies in mice have confirmed that changing the regulatory cell balance in immunopathological disease states by adoptive transfer of regulatory Th1 or Th2 cells can be therapeutic. As described previously (see, U.S. application Ser. No. 08/506,668, converted to U.S. provisional application Serial No. 60/044,693, now abandoned; co-pending U.S. applications Ser. Nos. 08/700,565, 09/127,411, 09/127,142, 09/127,138, 09/127,141, 09/824,906, and published International PCT application No. WO 97/05239), regulating the Th1/Th2 cell balance is therapeutic.

For example, adoptively transferred Th2 cells suppress Th1-mediated disease in animal models of uveoretinitis (Saoudi et al. (1993) European Journal of Immunology 23:3096), IDDM (Han et al. (1996) Journal of Autoimmunity 9:331), multiple sclerosis (Nicholson et al. (1995) Immunity 3:397) and allotransplantation (Fowler et al. (1994) Blood 84:3540; Fowler et al. (1994) Progress in Clinical and Biological Research 389:533). Adoptive transfer of Th1 clones protects animals against infection with the protozoan Leishmania major (Powrie et al. (1993) European Journal of Immunology 23:3043), genital infection with chlamydia trachomatis and murine candidiasis (Romani et al. (1991) Infection and Immunity 59:4647; Igietseme et al. (1999) Cancer Immunology, Immunotherapy 48:204; Ramsey et al. (1993) Regional Immunology 5:317).

Regulating Th1/Th2 balance in cancer is also therapeutic. The critical role for Th1-dominant immunity in tumor immunology is known (see, e.g., Nishimura et al. (2000) Cancer Chemotherapy and Pharmacology 46 Suppl:S52, for a review). For example, an extract from Mycobacterium tuberculosis, designated Z-100, restores Th1/Th2 balance in tumor-bearing mice (Oka et al. (1999) Immunology Letters 70:109) and inhibits pulmonary metastasis of B16 melanoma (Kobayashi et al. (1997) Anti-Cancer Drugs 8:15691) and Lewis lung carcinoma (Emori et al. (1996) Biotherapy 9:249). Z-100 is also a useful adjuvant in the treatment of oral cancer (Okutomi et al. (2000) Gan To Kagaku Ryoho (japanese Journal of Cancer and Chemotherapy) 27:65). Alkylating agents such as cyclophosphamide can cause complete remissions in tumor-bearing mice by changing the immune status from Th2-dominance to Th1-dominance (Inagawa et al. (1998) Anticancer Research 18:3957; Li et al. (1998) Journal of Surgical Oncology 67:221). Treatment of mice bearing large MOPC-315 tumors with L-phenylalanine mustard therapy stimulates anti-tumor immunity by causing a shift in cytokine production from Th2 to Th1 (Gorelik et al. (1994) Immunology, Immunotherapy 39:117). The streptococcal preparation, OK-432, inducea a Th1-dominate state in mice (Fujimoto et al. (1997) Journal of Immunology 158:5619; Okamoto et al. (1997) International Journal of Cancer 70:598) and has a potent anti-tumor effect in humans (Kitahara et al. (1996) Journal of Laryngology and Otology 110:449). The immunomodulator, AS101, has anti-tumor properties mediated through the stimulation of Th1 cytokine release in subjects and tumor-bearing mice (Sredni et al. (1996) Journal of the National Cancer Institute 88:1276; Sredni et al. (1996) International Journal of Cancer 65:97; Sredni et al. (1995) Journal of Clinical Oncology 13:2342). Intravesical Bacillus Calmette-Guerin (BCG) immunotherapy is an optimal choice for treatment of aggressive superficial bladder cancer, with a 70% response rate. The mechanism of BCG's therapeutic effect is through the stimulation of an increase in the production of Th1 cytokines (Thanhäuser et al. (1995) Cancer Immunology, Immunotherapy 40:103). Mistletoe extracts have antitumor activity in mice (Weber et al. (1998) Arzneimittel-Forschung 48:497; Yoon et al. (1998) International Journal of Immunopharmacology 20:163) and have positive affects on the quality of life in advanced cancer subjects (Friess et al. (1996) Anticancer Research 16:915). These effects appear to be mediated by stimulation of Th1 cytokines.

TCR-based vaccines that induce a Th1 immune response provide tumor protection in mice (Wong et al. (1999) Journal of Immunology 162:2251). Induction of antitumor CTL in mice bearing p53+ tumors is associated with measurable defects in the function of dendritic cells (DC). Tumor progression is associated with change of the balance Th1/Th2 cells in favor of the Th2-like cytokine profile, while effective immunization is associated with a shift to the Th1 phenotype (Gabrilovich et al. (1996) Cellular Immunology 170:111). DC-induced antitumor effects are completely blocked by coadministration of neutralizing monoclonal antibody directed against Th1-associated cytokines (such as IL-12, tumor necrosis factor alpha and IFN-γ) (Zitvogel et al. (1996) Journal of Experimental Medicine 183:87). Down-regulation of the Th2 response in tumor-bearing mice by treatment with anti-IL-4 mAb significantly suppresses growth of RENCA (murine renal cell carcinoma) tumors (Takeuchi et al. (1997) Cancer Immunology, Immunotherapy 43:375), while IL-2 gene transfected RENCA cells mediate tumor rejection (Hara et al. (1996) Japanese Journal of Cancer Research 87:724).

Adoptive immunotherapy experiments have also demonstrated the therapeutic utility of inducing Th1-dominated immunity to treat viral diseases. For example, transfer of influenza-specific Th1 cells was protective against influenza infection, while Th2 infusion failed to induce protection (Graham et al. (1994) Journal of Experimental Medicine 180:1273).

Polyclonal Th1 Cells for Adoptive Immunotherapy

Animal models of cancer have demonstrated that optimally prepared, adoptively transferred CD4+ T cells can reject established tumors with great efficiency even when targeted tumor cells express no MHC Class II molecules, implying that recognition of tumor antigen (Ag) occurs via MHC Class II-expressing host antigen-presenting cells (APC) within the tumor. Because consequent rejection also excludes Ag-specific contact between CD4+ T cells and MHC Class II negative tumor cells, the most critical CD4+ T-cell-mediated event is likely Th1 cytokine release, resulting in an accumulation and activation of accessory cells such as tumoricidal macrophages and lymphokine-activated killer cells (Cohen et al. (2000) Critical Reviews in Immunology 20:17).

Polyclonal Th1 cells, by virtue of their cytokine release, provide a general immune system boost that could deviate an on-going immune response from Th2 to Th1. This is supported by the observation that polyclonal Th1 cells administered to mice with non-immunogenic tumors results in rejection of 60-90% of the tumors. Animals cured by this treatment developed a tumor-specific memory and were capable of rejecting rechallenges with the same tumor (Saxton et al. (1997) Blood 89:2529). Similarly, co-injection of a PPD-specific Th1 clone, not capable of being activated by the tumor, and PPD antigen in a murine metastatic tumor model resulted in anti-metastatic effects and anti-tumor activity (Shinomiya et al. (1995)Immunobiology 193:439). Activated and expanded L-selectin-CD4+ T cells demonstrating a Th1 cytokine profile have also been shown to have excellent antitumor efficacy in mice (To et al. (2000) Laryngoscope 110:1648).

Accordingly, polyclonal Th1 cells that are activated ex vivo are desired for adoptive immunotherapy of human disease. Infusion of activated polyclonal Th1 cells could act by either suppressing the production of Th2 cytokines from all sources in subjects or by causing a shift in the immune response from Th2 to Th1. These cells by virtue of their ex vivo activation would not be under the influence of disease-specific immunosuppressive cytokines. Infused polyclonal Th1 cells also mediate enhanced cellular immune function through cell-to-cell contact, such as via the expression of CD40L which acts to cause macrophages to produce IL-12, a known immuno-enhancing cytokine. Polyclonal Th1 cells, via the production of IL-2, also act by stimulating semi-activated effector cells (NK, CTL) in tumor lesions. A combination of these known and other unknown mechanisms will result in enhanced cellular immunity after activated polyclonal Th1 cell infusion. The natural immune system under the influence of Th1 cytokines shifts the immune response to Th1 through the recognition of unknown disease associated antigens.

It is also desirable to have a process which can reproducibly produce highly pure populations of activated polyclonal Th1 cells for infusion. It is important that the expanded cells be highly pure to prevent the infusion of more Th2 cells than were removed from the subject. The infusion of Th2 cells could make the disease worse and can also, due to their cross-regulatory effect, inactivate the beneficial Th1 cells.

It is also desirable that the cells for infusion be processed in serum-free medium to avoid the expense and regulatory concerns associated with production of biological products for human infusion in serum containing media. Autologous serum supplementation is also not desired due to the immunosuppressive factors resident in the serum of subjects with cancer and other Th2 dominated diseases.

Cell Trafficking

Activated polyclonal Th1 cells are efficacious due to the bystander effect of the proinflammatory cytokines they produce. In order for polyclonal Th1 cells to have a therapeutic effect, it is advantageous for them to produce their proinflammatory cytokines in the vicinity of the tumor or other disease lesions. This requires that the cells traffic to the sites of inflammation or to tumors following their infusion.

In adoptive cell therapy protocols it is desirable to develop a population of cells that have the ability to traffic to tumors or sites of inflammation where they can influence the local environment. Ex vivo cell processing of cells for adoptive transfer does not always lead to the production of cells that traffic to tumor lesions. Previous studies with gene marked TIL cells and peripheral blood lymphocytes (PBL) show that these adoptively transferred cells are detected circulating in the peripheral blood for up to 99 days after infusion. No convincing pattern of preferential trafficking of TIL versus PBL to tumor was noted (Economou et al. (1996) Journal of Clinical Investigation 97:515). The methods provided herein result in Th1 memory cells that traffic to tumors and sites of inflammation.

It is known that T-cells that express an activated memory phenotype will selectively accumulate within tumor lesions and other sites of inflammation. Activated memory T-cells have a CD3+, CD25+, CD45RO+, CD62L^{lo} phenotype. It is also known that the expression of CD44 can enhance the ability of cells to infiltrate tissues. The methods provided herein produce highly pure populations of Th1 cells that have an activated memory phenotype. The methods herein involve purification of Th1 cells precursors, such as CD4+, CD45RA+ T-cells, and their subsequent differentiation and expansion. The methods are also designed to minimize or substantially or completely eliminate any Th2 cell contamination in the final product.

High Toxicity of Prior Art Adoptive Immunotherapy

The toxicity of adoptive immunotherapy treatments has been associated with the use of the growth factor, IL-2. Exogenous IL-2, also known as “T-cell Growth Factor”, is used in adoptive immunotherapy for the differentiation of immune cells into cytotoxic effector cells and for the ex-vivo expansion of T-cells. The exposure of immune cells to exogenous IL-2 makes them dependent upon the continued presence of IL-2 to maintain their viability and function. This has necessitated the co-infusion of IL-2 with the cells in prior art adoptive immunotherapy protocols. The systemic administration of IL-2 results in severe and often life-threatening toxicity.

While toxic, it is known that even non-therapeutic doses of IL-2 can significantly enhance the therapeutic efficacy of infused immune cells by inducing in-vivo proliferation and prolonged survival. Therefore, IL-2 is infused routinely in adoptive immunotherapy methods. The reason IL-2, even in non-therapeutic doses, enhances the efficacy of infused cells is because prior art adoptive immunotherapy methods produce cells that are not optimally activated. Non-therapeutic doses of IL-2 tend to increase the activation state of the cells providing therapeutic benefit. Infusion of IL-2 and cells together complicates the ability to obtain regulatory approval of the cell infusion as a biological drug, as it is difficult to determine the contribution of the cells separate from the contribution of IL-2. Accordingly, it is desirable to eliminate the need for IL-2 in the differentiation and expansion phases of adoptive immunotherapy, as well as in the infusion phase. Cells resulting from the methods are in a highly activated state (CD25+) and produce significant amounts of cytokine without further stimulation.

C. Methods for Producing Highly Pure, Activated Th1 Cells

Methods for consistently producing a population of highly pure, activated, polyclonal memory Th1 cells from a subject blood sample in the absence of any exogenous growth or differentiation factors (such as IL-2 or IFN-γ) for use in adoptive immunotherapy are provided. The methods provided herein include the steps of: (i) the collection of source material from a subject; (ii) the purification of T-cells from the source material; (iii) the frequent (every 2-3 days) activation of the purified T-cells and typically repeated (a minimum of 3 times); and optionally (iv) the reinfusion of the resulting cells into the same subject.

1. Source Cell Collection

In practicing a method provided herein, a starting population of mononuclear cells is collected from a subject by leukapheresis, in order to obtain the greatest starting cell population number. This is the source material. A population of CD3+ T-cells, generally CD4+ cells, is then purified from the source population of mononuclear cells. Purities should be in excess of 90%. These are the starting population of cells. The CD4+ cells can be purified by positive selection as more fully explained below. In subjects with large numbers of Th2 cells resident in the memory cell population (CD45RO+), the CD4+ cells can be further purified in order to obtain a starting population of only naive CD4+ cells. This is accomplished by purging the CD4+ cells of CD45RO+ cells. Purified CD4+ cells will express CD45RA+ and CD62L^{hi} surface antigens and produce IL-2 upon activation. CD4+ cell populations purified and activated as provided herein contain few, if any, IL-4 producers and also fail to initially make substantial amounts of IFN-γ. The methods provided herein are capable of producing a pure population of activated Th1 memory cells from a starting population of CD4+ cells, as well as capable of enhancing the population of activated Th1 memory cells from starting populations of CD3+ cells and CD4+, CD45RO+ cells. It is known that CD4+ cells can develop into cells that principally produce IL-4 or IFN-γ upon restimulation. All prior art methods teach the use of exogenous cytokines to cause this differentiation ex vivo.

2. Initial Activation

The starting cells must undergo an activation step in order to develop into Th1 memory cells. Generally it is known that CD4+ cells can be activated by antigen presented on MHC Class II molecules or polyclonal stimulants such as Con A, PMA or anti-CD3. For purposes of herein, an exemplary method of activation method is immobilized anti-CD3/anti-CD28 mAb costimulation. In order to assure the differentiation of Th1 cells after activation, the concentration of IL-4 at the time of activation has to be extremely low or even non-existent. IL-4 is known to have a profound effect on the ability of the CD4+ cells to differentiate into Th2 cells. For example, activation of CD4+ cells in the presence of IL-4 concentrations of as little as 50 pg/ml is enough to cause the population of Th2 cells in the culture to increase greater than 100-fold. This increase is known to be due to differentiation of CD4+ cells into Th2 cells and not the expansion of pre-existing Th2 cells. Therefore, it is important to assure that the starting population of cells collected for the purpose of ex-vivo differentiation of Th1 cells are purged of all cells that are producing IL-4. Failure to purge IL-4 producing cells prior to the initial activation will result in Th2 cell contamination of the final product.

3. Initial Purification

Because the starting population of cells must be activated in the absence of IL-4 in order to prevent Th2 differentiation, the cellular sources of IL-4 must be first purged from the starting culture. The cellular source of the early burst of IL-4 that drives Th2 differentiation in-vivo has not been conclusively identified. Therefore, the exact cell types necessary to purge from the starting culture is not clear. Among the cell types that should be purged are CD117+ granulocytes, basophils, NK cells, and NK1.1 T-cells, which are sources of IL-4 (see, Wang et al. (1999) Clinical Immunology 90:47; Poorafshar et al. (2000) European Journal of Immunology 30:2660; Singh et al. (1999) Journal of Immunology 163:2373; Leite-De-Moraes et al. (1998) European Journal of Immunology 28:1507; Poynter et al. (1997) Cellular Immunology 179:22). So at least these subsets of cell are purged from the starting culture.

Immune cell subsets are commonly purged by using monoclonal antibodies specific for unique cell surface molecules on the target cells. To isolate cells, they can be indirectly stained with specific biotinylated antibody and passed through a avidin-coated column (Handgretinger et al. (1994) Journal of Clinical Laboratory Analysis 8:443) or the antibodies can be immobilized on immunomagnetic beads or particles directly, mixed with the cells and placed under a magnetic field (Mantovani et al. (1989) Bollettino—Societa Italiana Biologia Sperimentale 65:967; Jacobs et al. (1993) Research in Immunology 144:141; Partington et al. (1999) Journal of Immunological Methods 223:195). Alternatively, the cells can be labeled with the monoclonal antibody and mixed with immunomagnetic particles coated with species-specific antibodies that bind to the monoclonal antibody specific for the cell surface marker (indirect method) (Hansel et al. (1989) Journal of Immunological Methods 122:97). Immobilizing the monoclonal antibody to a solid surface, such as a culture flask (panning) can also be used (Prince et al. (1993) Journal of Immunological Methods 165:139), as well as florescent-activated cell sorting techniques.

Negative selection can be performed with a cocktail of monoclonal antibodies (mAb) specific for cell surface markers that are exclusively expressed on the unwanted cells. For example, for purging the cells herein, a cocktail containing mAbs to CD19 (B-cells), CD56 (NK cells), CD14 (monocytes/macrophages) and CD8 (cytotoxic T-cells) was used to obtain a population of pure CD4 cells by negative selection. This cocktail when used with immunomagnetic beads results in a pure population of CD4+ cells (>95%) when the cells are derived from normal donors.

For purposes herein, however, negative selection purification techniques are not desirable for purification of the source cells. Negative selection leads to an unknown starting population of cells that can negatively affect the purity of the final product. Subjects with immunologically-mediated diseases, and cancer subjects in particular, present with a wide variety of hematological profiles. Subject blood can have many immature cells with altered surface expression so it is difficult to define a monoclonal antibody cocktail that can purge all unwanted cells from a mononuclear cell sample from every subject. These unidentified cells can contaminate the starting cell population. The same mAb cocktail that results in a pure population of CD4 cells from normal donors, when used on blood samples from cancer subjects, results in CD4 cells with very poor purity (only 30-60% CD4+). The poor purity of the starting population of cells prevents the generation of a high purity final product of Th1 cells.

Therefore, in embodiments herein a positive selection protocol is used in order to isolate pure populations of CD4 cells from subject blood. Positive selection allows the retention of only the desired CD4+ cells, while all the unwanted contaminating cells, of known and unknown phenotypes, are purged from the culture. A method for positive selection is to use an anti-CD4 mAb conjugated to immunomagnetic beads or magnetic particles in order to positively select CD4+ cells from the source subject blood samples.

Purification of source cells is rarely used in prior adoptive immunotherapy methods and when it is used, negative selection protocols have been preferred. Positive selection is not often used to purify immune cell subsets due to the difficulty of removing the selected cells from the beads after the selection. Physically removing the cells from the beads by gentle agitation results in very pure CD4 cells (greater than 95% CD4+), it also results in a lower yield than negative selection techniques (yields of 50-60% compared to greater than 70% using positive selection). Another problem with positive selection is that significant numbers of cells retain mAb on their CD4 receptors or internalize their CD4 receptors after selection, making it difficult to access the purity of the cells by FACS. This can be solved by waiting 24-48 h before analysis or by staining for CD3+, CD8− cells as an indirect determination of CD4+ cells.

Another reason why positive selection has not been used to purify T-cells, especially CD4+ T-cells, from source material is that such techniques have technical problems when being applied to source material derived from cancer subjects. The positive selection of CD4+ cells directly from mononuclear cells isolated from cancer subjects often lead to a massive loss of viability of the selected CD4 cells. This does not occur when the same positive selection techniques are applied to source material from normal donors. Some macrophages are known to express the CD4 surface marker, it appears that the purification process activates these macrophages causing them to produce a substance that is lethal to CD4+ T-cells. Since cancer subjects have been exposed to many different chemotherapy drugs and radiation treatments, this could predispose the macrophages to produce a lethal substance upon ligation of the CD4 molecule. Accordingly, when practicing the methods herein with cancer subjects, the macrophage component of the source cell population should be minimized prior to the CD4 positive selection step.

An exemplary method to reduce the macrophage population is to first incubate the collected mononuclear cells overnight on plastic. This takes advantage of the well known property of macrophages to adhere to a surface. The next morning, the non-adherent fraction of cells can be collected and subjected to positive selection of CD4 cells. Another method is to pass the mononuclear cells through a column of nylon wool prior to CD4 positive selection. Macrophages attach to the nylon wool fibers and are thus removed from the culture. The use of macrophage-specific mAbs and complement can also be used.

Prior removal of the adherent fraction of mononuclear cells enabled CD4 cells to be positively selected from cancer subject mononuclear blood samples without loss of viability.

4. Differentiation of Th1 Cells

Activation in the presence of IFN-γ and the absence of IL-4 is generally believed to be required to cause CD4+ to differentiate into Th1 cells. Advantageously, methods provided herein do not require the addition of any cytokines. Also, the methods do not require the presence of macrophages for differentiation, which play a critical role in directing CD4+ cells to differentiate into Th1 or Th2 cells. Macrophages, however, are short-lived in cultures, and thus limit the applicability of methods and compositions that rely macrophages for differentiation. The methods herein, thus, avoid this.

The initial activation of purified CD4+ cells with immobilized anti-CD3 and anti-CD28 induces the cells to produce IL-2 and no IFN-γ. Without further stimulation, the cells expand and differentiate into mixed populations of Th1 and Th2 cells. When the CD4 cells are derived from cancer subject blood, there is sometimes production of detectable amounts of IL-4 in the cultures after the initial activation with anti-CD3/anti-CD28. CD4 cells positively selected after depletion of non-adherent monocytes are known to produce IL-4 (Stanciu et al. (1996) J. Immunolog. Methods 187:107-115).

When IL-4 is detected after the initial activation, a significant amount of the IL-4 was found to be produced by the memory CD4+, CD45RO+ subpopulation of the starting cells. Others have also identified memory cells as a source of IL-4 (Sasama et al. (1998) International Archives of Allergy and Immunology 117:255).

Because of the Th1/Th2 imbalance in cancer subjects and in other subjects with diseases in which the Th2 phenotype predominates, the memory cell subset of CD4+ cells is enriched in IL-4 producing cells. Therefore, it may be necessary to also purge the CD45RO+ cells from the starting cells to enhance the purity of the final population of Th1 cells. The necessity for this purging step can be determined empirically for a particular subject or disease state, or the step can be routinely included to ensure that such cells, if present, are eliminated.

As described herein, the method provided herein that employs frequent activation with immobilized anti-CD3/anti-CD28 can cause such high amounts of endogenous IFN-γ production from the culture that any contaminating cells with the capacity to produce IL-4 are inhibited. Therefore, while small amounts of IL-4 may be detectable in the early activation steps, IL-4 production becomes negligible after several rounds of activation with anti-CD3/anti-CD28. Therefore, it is rarely required that the CD45RO+ population needs to be purged from the starting cells, even when the source cells are derived from cancer subjects.

If the CD45RO purge step is performed, additional technical issues need to be addressed. After collection of mononuclear cells by leukapheresis, if the CD4 positive selection is performed prior to the CD45RO purge, there is a significant loss of yield. This is because residual mAb on CD4 cells causes CD4 cells to be purged with the CD45RO cells. For this reason, in one embodiment the macrophage fraction removed first, and the CD45RO+ cells are purged by negative selection followed by positive selection for CD4+ cells. This results in a pure population of viable CD4+, CD45RA+ naive T-cells (pTh cells).

When processing cancer subject blood, the CD45RO purge step followed by the CD4 positive selection often results in viable cells, even without the macrophage reduction step. This is due to the significant loss of adherent cells during the CD45RO negative selection process. For consistent production of Th1 cells from a variety of subject blood, the purge the macrophage population prior to purification of the CD4 or pTh cells should be performed.

Unlike prior methods, the purified pTh or CD4 cells can be caused to differentiate into pure populations of Th1 cells without addition of exogenous cytokines. Activation of pTh cells by a variety of methods, including anti-CD3/anti-CD28, is known to result in the differentiation of Th2 cells. Naive CD4+ cells are a significant source of IL-4 (Noben-Trauth et al. (2000) Journal of Immunology 165:3620; Demeure et al. (1995) European Journal of Immunology 25:2722). It has been reported that almost every single naive human CD4 T cell primed and expanded in the absence of exogenous IL-4 releases sufficient autocrine IL-4 to support differentiation into Th2 cells (Yang et al. (1995) European Journal of Immunology 25:3517).

It was found herein, however, that when pTh cells or CD4+ cells were repeatedly and frequently (about every 2-3 days) activated with anti-CD3/anti-CD28 that they do not produce IL-4. Upon each stimulation, the cells produced increasing amounts of IFN-γ. In particular, it is shown herein, that when pTh cells or CD4 cells are repeatedly (minimum of 3 times) and frequently (every 2-3 days) activated with anti-CD3/anti-CD28 that they do not produce IL-4. Upon each stimulation, the cells produce increasing amounts of IFN-γ. The repeated activation causes such large amounts of IFN-γ to be produced that it compensates for a poor quality initial purification and still resulting in highly pure Th1 memory cells at the end of the process. The large amounts of IFN-γ produced into the culture act to inhibit any production of IL-4 by contaminating cells. Reactivation at a frequency of every 2-3 days for a period of about 9-14 days consistently results in the differentiation of highly pure populations of Th1 memory cells even if the starting population is CD3+ T-cells (CD4+ cells contaminated with CD8+ cells; see, EXAMPLES).

5. Expansion Without IL-2

CD4 cells purified from cancer subjects and activated with immobilized anti-CD3/anti-CD28 do not expand efficiently without the addition of exogenous IL-2. It is known that T-cells from normal donors expand without exogenous IL-2 after being stimulated with anti-CD3/anti-CD28 (see, (Ledbetter et al. (1985) Journal of Immunology 135:2331; Levine et al. (1997) Transplantation Proceedings 29:2028). When the cells are derived from cancer subject blood, however, the addition of exogenous IL-2 is required to create optimal growth conditions for anti-CD3/anti-CD28 activated T-cells from cancer subjects (Garlie et al. (1999) Journal of Immunotherapy 22:336). There are no reports of successful expansion of cancer-derived T-cells without the use of exogenous IL-2.

Source cells from cancer subjects were found to contain significant amounts of TGF-beta. TGF-beta is known to down regulate T-cell proliferation. Significant amounts of the TGF-beta appear to originate from platelets, which are a known source of TGF-beta (Werz et al. (1996) Pharmazie 51:893). Processing of subject blood causes the release of significant amounts of TGF-beta presumably from the platelets, whereas TGF-beta release is not evident in cultures of processed normal blood. It is not known why the platelets from cancer subjects release TGF-beta during processing, but it may be related to the effect of radiation and chemotherapeutic drugs on the fragility of the platelets. Increased plasma levels of TGF-beta have been reported in subjects with cancer (Jiang et al. (1995) Acta Haematologica 94:1).

Accordingly, the platelet population is reduced in the collected mononuclear cells prior to any processing. This can be achieved, for example, by centrifuging the collected mononuclear cells, such as centrifugation for about 2-5 minutes at 150×g, followed by purging the platelet rich supernatant. Purging platelets from the starting population of mononuclear cells permits cancer subject T-cells to be efficiently expanded with anti-CD3/anti-CD28 mAb without the requirement for exogenous IL-2 addition.

The isolation of pure CD4+ T-cells from subject blood, and the subsequent activation of the cells repeatedly with immobilized anti-CD3 and anti-CD28 mAb results in the expansion of these cells without exogenous cytokines and consistently generates activated Th1 memory cells with high purity. These resulting Th1 memory cells produce large amounts of IFN-γ and no detectable IL-4 and express an activated memory phenotype (CD3+, CD4+, CD45RO+, CD62L−, CD25+, CD44+).

D. Practice of the Therapeutic Methods

The therapeutic methods herein are designed to produce compositions containing clinically relevant (at least 10⁹, preferably 10¹⁰ cells or more, generally in a volume of a liter, 500 mls, 200 mls, 100 mls or less) populations of polypclonal memory Th1 cells for infusion for treatment of the diseases or conditions characterized by suppression of the cellular immune response, by over-expression of the humoral immune response, excess Th2 activity or a lack or decreased Th1 activity. The methods herein do not rely or use any agents for expansion or differentiation that must be present after expansion to maintain cell viability or activity.

The compositions contain highly (greater than 70%, 80%, 90% or more of the cells) pure populations of polyclonal memory Th1 cells. Such compositions are used therapeutically for treatment of the diseases, such as cancer, infectious diseases, allergic diseases and other diseases or conditions characterized by suppression of the cellular immune response, by over-expression of the humoral immune response, excess Th2 activity or a lack or decreased Th1 activity.

Administration

The compositions of cell can be administered by any suitable means, including, but not limited to, intravenously, parenterally, or locally. The particular mode selected will depend upon the particular treatment and trafficking of the cells. Typically, about 10¹⁰-10¹¹ cells can be administered in a volume of a 50 ml to 1 liter, 50 ml to 250 ml, 50 ml to 150, and typically 100 ml. The volume will depend upon the disorder treated and the route of administration. The cells can be administered in a single dose or in several doses over selected time intervals in order to titrate the dose.

Vaccines

Also provided herein vaccines that are a combination of the cells produced herein and an immunizing antigen, and methods of vaccinating by co-infusing, either sequentially or simultaneously, the cells produced herein and an immunizing antigen, such as tumor-associated antigens, viral antigens, bacterial antigens and other any such antigens. The vaccines can be immunoprotective or can ameliorate symptoms of a disease or treat such disease, for example, by increasing an immune response such as the immune response against tumor-associated antigens.

The cells produced by the methods provided herein can be co-infused with an antigen or the antigen and cells can be administered separately, sequentially or intermittently.

The following examples are included for illustrative purposes only and are not intended to limit the scope of the invention.

### EXAMPLE 1

Materials and Methods

A. Isolation of Human Lymphocytes.

Samples of buffy coats or leukapheresis products from normal donors and EDTA-preserved blood samples from advanced cancer subjects with a variety of indications and prior treatments were used. Human peripheral blood lymphocytes (PBMC) were isolated using a density gradient centrifugation procedure.

B. Characterization of PBMC Samples

Purified PBMC samples were characterized by immuno-phenotyping using flow cytometry. Briefly, cells were incubated with fluorochrome-labeled antibodies in the dark for 30 min., washed of excess antibodies and analyzed on FACSCalibur flow cytometer (BD Biosciences). Results of the analysis were expressed as percentages of total lymphocytes, monocytes, granulocytes, and also subsets of lymphocytes: B-cells, cytotoxic T lymphocytes, CD4 positive T-helpers, and NK cells. The subset of CD4 positive T cells was analyzed for the ratio between naive CD45RA positive cells and CD45RA negative memory cells.

C. Cytokine Profiling

To determine the ability of freshly purified CD4 positive cells to express IFN-γ and IL-4 an intra-cellular cytokine (ICC) staining procedure using an Internal Cellular Cytokine (ICC) kit (BioErgonomics, St. Paul, Minn.) was performed. According to the manufacturer's recommendation, PBMC were stimulated for 20 h in T-cell activation medium, stained first by surface anti-CD4 antibodies, fixed, permeated and then stained with intracellular anti-IFN-γ and anti-IL-4 antibodies. Samples were analyzed by flow cytometry and results were presented as percentages of IFN-γ and IL-4 expressing cells in CD4 positive T cells subset.

D. Isolation of T-cell Subpopulations

Isolation of specific T-cell subpopulations was performed using two different techniques: sort by flow cytometry on FACSCalibur and sort by combination of positive and negative immunomagnetic selection on AutoMacs (Miltenyi, Germany). To obtain cell samples with high purity, sort by flow cytometry was done. Briefly 4×10⁷ of PBMC were stained with anti-CD4 antibodies alone or in combination with anti-CD45RO antibodies, labeled with the corresponding fluorochrome. Subsets of CD4-positive, CD4-positive/CD45RO-negative and CD4-positive/CD45RO-positive cells were collected by sorting and used for expansion experiments. To obtain better yields with 5-10% lower purities, separation for further applications used immunomagnetic selection.

According to the manufacturer's recommendation, up to 2×10⁸ cells were incubated with anti-CD4 antibodies conjugated directly to magnetic microbeads and separated on magnetic columns. If needed, the second round of selection was performed using mouse anti-CD45RO antibodies in complex with goat anti-mouse antibodies conjugated to microbeads.

E. Activation of Cells

Sorted cells were plated into cell culture plates at starting concentrations of 1×10⁵ to 3×10⁵ cells/ml using ex vivo serum free cell culture medium (X-VIVO-15 from BioWhittaker) without supplementation. The cells were cultured for 12 days and were repeatedly activated using a combination of CD3/CD28 antibodies conjugated to magnetic beads (T-cell Expander, Dynal) every 3 days, starting from the day of sort.

Initial cell activation was performed using 3:1 ratio between magnetic beads and sorted cells. For re-stimulation, an amount of beads equal to the amount of cells in the culture determined by hand cell count was used. On day 13, 14 or 15 expanded cell cultures were harvested. The cells were counted cells (manual hand count) and the final product was characterized.

F. Phenotyping

For characterization of the final product, the phenotypes of harvested cells were determined, their ability to express IFN-γ and IL-4 by intra-cellular cytokine staining (ICC) and their production of IFN-γ, IL-2 and IL-4 (determined by ELISA in the cell culture supernatants of expanded cells before harvesting) were analyzed. Immunophenotyping and ICC experiments were performed as described above. ELISA assays were performed using ELISA kits (R&D, Minneapolis, Minn.) for IFN-γ, IL-2, IL-4, IL-10, IL-13, TNF-alpha according to manufacturer's recommendations.

### EXAMPLE 2

CD4+ cells purified from the peripheral blood of a cancer subject were divided in two groups: Group 1 were activated every 3 days for a period of 12 days and harvested on the 15th day. Group 2 were activated only once and harvested on the 15th day. Both groups of cells were then reactivated and incubated in the presence of IL-10 (100 pg/ml), IL-4 (50 pg/ml), IL-6 (100 pg/ml) and TGF-beta (100 pg/ml) to stimulate an immunosuppressive tumor environment. As a control, each a portion of each group of cells was activated in the absence of immunosuppressive cytokines. The production of IFN-γ was measured after 24 hours and expressed as production per 10⁶ cells per 24 hours.

| | |
|---|---|
| Group 1 | Group 2 |

| No Cytokines | Cytokines | No Cytokines | Cytokines |
|---|---|---|---|
| 24 pg/ml | 22 pg/ml | 2 pg/ml | 0.05 pg/ml |

These results indicate that cells produced by the methods provided herein are resistant to the immunosuppressive effects of cytokines that stimulate the intratumoral microenvironment. Cells subjected to a single activation produce 10-times less IFN-γ than the cells produced by the methods herein. Further, IFN-γ production is inhibited by the presence of immunosuppressive cytokines in these cells.

### EXAMPLE 3

Prior art methods using immobilized anti-CD3/anti-CD28 stimulation have not been successful in expanding T-cells derived from cancer subjects without IL-2 supplementation. By eliminating the sources of TGF-beta from the initial cultures, cancer subject T-cells could be efficiently expanded without IL-2. This experiment was designed to determine the effect IL-2 addition had on the phenotype of the resulting cells using a prior art single stimulation method compared with the repeated frequent stimulation method provided herein.

CD4+ cells from a cancer subject were purified by sorting on a flow cytometer. The resulting cells were cultured under the following conditions for 14 days: (Group 1) initial stimulation with anti-CD3/anti-CD28 with no IL-2; (Group 2) initial stimulation with anti-CD3/anti-CD28 with IL-2 (100 IU/ml); (Group 3) initial stimulation with anti-CD3/anti-CD28, no IL-2 and restimulation with anti-CD3/anti-CD28 every 3 days; and (Group 4) initial stimulation with anti-CD3/anti-CD28, IL-2 (100 IU/ml) and restimulation with anti-CD3/anti-CD28 every 3 days.

| | Group 1 | Group 2 | Group 3 | Group 4 |
|---|---|---|---|---|
| CD4 | 99.56% | 99.8% | 91.34% | 92.52% |
| CD45RA | 29.02% | 43.66% | 10.84% | 10.94% |
| CD45RO | 42.62% | 70.28% | 73.64% | 78.12% |
| CD62L | 46.24% | 52.76% | 1.91% | .093% |
| CD25 | 64.02% | 46.54% | 87.89% | 82.21% |
| CD44 | 99.94% | 99.94% | 92.91% | 89.14% |
| Internal IFN+ | 24.65% | 32.87% | 86.83% | 73.24% |
| Internal IL-4+ | 15.7% | 24.64% | 7.42% | 9.67% |
| IFN ELISA | 85.6 pg/ml | 105.9 pg/ml | 8773 pg/ml | 4401 pg/ml |
| IL-4 ELISA | <26 pg/ml | <26 pg/ml | <26 pg/ml | <26 pg/ml |
| Fold Expansion | 83 | 135 | 320 | 170 |

These results indicate that CD4+ cells derived from cancer subjects can be expanded with anti-CD3/anti-CD28 stimulation when the starting population is purged of platelets with or without the addition of IL-2. The addition of IL-2 also had little effect on the final phenotype of either group.

The repeated and frequent stimulation method enhances the ability of the cells to proliferate. These data also show that the repeat stimulation method results in a population of cells that has enhanced activation (CD25) and greatly enriched for IFN-γ production and IFN-γ internal staining. It is also relevant that the repeated and frequent stimulation method results in cells that have very low CD62L expression. These cells have a greater ability to infiltrate tumors and other sites of inflammation.

### EXAMPLE 4

CD4+, CD45RA+ cells purified from the peripheral blood of a cancer subject were stimulated every 3 days with anti-CD3/anti-CD28. The cells were harvested on day 14 and analyzed for internal expression of IFN-γ and IL-4.

| Day | % IFN-γ + internal stain | % IL-4 + internal stain |
|---|---|---|
| 0 | 3.78 | 1.89 |
| 14 | 99.35 | 2.42 |

These data indicate that the method provided herein can cause naive T-cells to differentiate into a highly pure population of Th1 cells.

### EXAMPLE 5

CD4+ cells were purified from a normal donor. The cells in Group 1 were stimulated with anti-CD3/anti-CD28 only once. The cells in Group 2 were stimulated every 3 days. Both groups were cultured for 14 days.

| | Group 1 | Group 2 |
|---|---|---|
| CD4 | 99.47 | 97.92 |
| CD45RA | 10.29% | 18.23% |
| CD45RO | 16.58% | 81.47% |
| CD62L | 46.97% | 1.92% |
| CD25 | 18.07% | 97.10% |
| CD44 | 99.52% | 99.08% |
| Internal IFN+ | 23.35% | 71.68% |
| Internal IL-4+ | 6.14% | 4.08% |
| IFN ELISA | 1651 pg/ml | 6870 pg/ml |
| IL-4 ELISA | 52 pg/ml | <26.1 pg/ml |

These data indicate that the process results in an enhanced population of activated (CD25+), memory (CD45RO+) Th1 cells compared to single stimulation methods.

### EXAMPLE 6

T-cells and T-cell subsets were purified from three different cancer subject PBMC by FACS. The blood was purified into four groups: (1) CD3+; (2) CD4+; (3) CD4+, CD45RO− and (4) CD4+, CD45RO+. The cells were stimulated every 3 days with immobilized anti-CD3/anti-CD28 mAb. The resulting cells were analyzed after 14 days of culture to assess their phenotypes.

| | | | CD4+, | CD4+, |
|---|---|---|---|---|
| Subject 1 | CD3+ | CD4+ | CD45RO− | CD45RO+ |
| CD4+ | 70.94% | 97.76% | 99.52% | 99.01% |
| CD8+ | 20.55% | 0.45% | 0.14% | 1.72% |
| CD45RA+ | 0.89% | 4.01% | 2.95% | 1.62% |
| CD45RO+ | 75.43% | 87.68% | 93.97% | 96.80% |
| CD62L+ | 2.49% | 1.87% | 9.72% | 13.75% |
| CD25+ | 78.98% | 96.02% | 92.97% | 96.08% |
| CD44+ | 79.47% | 99.20% | 99.78% | 99.42% |
| Internal IFN+ | 64.87% | 79.30% | 70.05% | 46.62% |
| Internal IL-4+ | 41.17% | 13.94% | 11.46% | 4.82% |
| IFN ELISA | 1612 pg/ml | 1092 pg/ml | 4332 pg/ml | 2664 pg/ml |
| IL-4 ELISA | <26 pg/ml | <26 pg/ml | <26 pg/ml | <26 pg/ml |
| IL-13 ELISA | 2810 pg/ml | 2227 pg/ml | 986 pg/ml | 703 pg/ml |
| TNF-α ELISA | 8055 pg/ml | 9000 pg/ml | 384 pg/ml | 359 pg/ml |
| IL-10 ELISA | 0 pg/ml | 0 pg/ml | 150 pg/ml | 128 pg/ml |
| | | | CD4+, | CD4+, |
| Subject 2 | CD3+ | CD4+ | CD45RO− | CD45RO+ |
| CD4+ | 70.15% | 98.35% | 97.51% | 96.09% |
| CD8+ | 23.53% | 0.42% | 0.19% | 3.65% |
| CD45RA+ | 0.93% | N.D. | 2.02% | 0.15% |
| CD45RO+ | 72.03% | N.D. | 96.47% | 94.06% |
| CD62L+ | 5.18% | N.D. | 20.89% | 13.22% |
| CD25+ | 67.37% | N.D. | 95.22% | 93.85% |
| CD44+ | 68.05% | N.D. | 96.24% | 95.74% |
| Internal IFN+ | 59.62% | 86.09% | 95.71% | 54.78% |
| Internal IL-4+ | 5.96% | 11.68% | 9.41% | 3.41% |
| IFN ELISA | 20,868 pg/ml | 25,514 pg/ml | 13,100 pg/ml | 1928 pg/ml |
| IL-4 ELISA | <26 pg/ml | <26 pg/ml | <26 pg/ml | <26 pg/ml |
| IL-13 ELISA | 325 pg/ml | 258 pg/ml | 978 pg/ml | 429 pg/ml |
| TNF-α ELISA | 1427 pg/ml | 1025 pg/ml | 2318 pg/ml | 2318 pg/ml |
| IL-10 ELISA | 380 pg/ml | 800 pg/ml | 320 pg/ml | 1000 pg/ml |
| | | | CD4+, | CD4+, |
| Subject 3 | | CD4+ | CD45RO | CD45RO+ |
| CD4+ | N.D. | 98.56% | 97.56% | 98.78% |
| CD8+ | N.D. | 0.07% | 1.75% | 0.16% |
| CD45RA+ | N.D. | 5.17% | 6.27% | 10.40% |
| CD45RO+ | N.D. | 96.60% | 97.36% | 96.00% |
| CD62L | N.D. | 1.30% | 5.55% | 5.21% |
| CD25+ | N.D. | 96.67% | 94.55% | 95.50% |
| CD44+ | N.D. | 99.67% | 97.60% | 99.52% |
| Internal IFN+ | N.D. | 86.63% | 73.45% | 82.03% |
| Internal IL-4+ | N.D. | 2.56% | 4.95% | 3.78% |
| IFN ELISA | N.D. | 4138 pg/ml | 2998 pg/ml | 2798 pg/ml |
| IL-4 ELISA | N.D. | <26 pg/ml | <26 pg/ml | <26 pg/ml |
| IL-13 ELISA | N.D. | 4034 pg/ml | 1746 pg/ml | 679 pg/ml |
| TNF-alpha | N.D. | 2287 pg/ml | 543 pg/ml | 846 pg/ml |
| ELISA | | | | |
| IL-10 ELISA | N.D. | 120 pg/ml | 380 pg/ml | 115 pg/ml |

These data indicate that methods herein generate enhanced populations of activated Th1 memory cells from subject blood with or without purification of T-cell subsets.

### EXAMPLE 7

The following data demonstrate the consistency in compositions of resulting cells produced by the methods provided herein from samples from eight different cancer subjects and 8 different normal subjects.

| Cancer Donors: | | | | | | | | |
|---|---|---|---|---|---|---|---|---|
| Initial phenotype | | | Harvest phenotype | | | | | |
| | Total | | (day 14) | | | | | |
| | CD4 | CD4/CD45RO | CD4 | CD4/CD45RO | CD4/62L low | CD4/CD25 | CD4/CD44 | %IFN-γ/IL-4 |
| 1 | 4.6% | 1.9% | 99% | 96.6% | 98.7 | 96.6 | 99.67 | 86.6/2.5 |
| 2 | 10% | 4.6% | 98.2 | 96.47 | 79.1% | 95.2 | 96.2 | 86.1/11.6 |
| 3 | 6.8% | 4.1% | 96 | 94.8 | 80.1 | 86.7 | 93.7 | 84.9/5.6 |
| 4 | 9.5 | 8.0 | 99 | 98.1 | 98.5 | 93.7 | 99.1 | 75.4/4.7 |
| 5 | 47.3 | 25.6 | 92 | 87.6 | 98.1 | 96.2 | 99.7 | 79.3/13.9 |
| 6 | 14.1 | 6.9 | 99 | 89.2 | 97.5 | 98.6 | 99.6 | 77.7/16.2 |
| 7 | 31 | 12 | 99 | 93.3 | 94.1 | 87.2 | 98.2 | 92.6/10.3 |
| 8 | 7.2 | 6.7 | 95 | 93.2 | 71.2 | 97.6 | 97.8 | 94.3/6.4 |
| Normal Donors: | | | | | | | | |
| Initial phenotype | | | Harvest phenotype | | | | | |
| | Total | | (day 14) | | | | | |
| | CD4 | CD4/CD45RO | CD4 | CD4/CD45RO | CD4/62L (−) | CD4/CD25 | CD4/CD44 | %IFN-γ/IL-4 |
| 9 | 33% | 23% | 94.6 | 94.5 | 72.1 | 87.23 | 99.9 | 82.1/0.9 |
| 10 | 35.8 | 19.3 | 98.2 | 98.2 | 96.4 | 07.47 | 99.8 | 97.1/3.7 |
| 11 | 27.5 | 12.1 | 99.1 | 99.4 | 91.2 | 98.6 | 99.4 | 71.9/5.1 |
| 12 | 6.4 | 3.4 | 97 | 89.7 | 98.3 | 92.98 | 93.7 | 73.8/13.3 |
| 13 | 23 | 15 | 99 | 88.1 | 99.7 | 97.7 | 98.4 | 90.2/12.2 |
| 14 | 35 | 19 | 99.9 | 99.9 | 70.1 | 98.1 | 91.4 | 86.4/7.2 |
| 15 | 33 | 18 | 99.9 | 99.9 | 82.3 | 93.8 | 92.9 | 80.3/11.2 |
| 16 | 29 | 18 | 95 | 94.5 | 85.5 | 90.1 | 94.6 | 94.6/3.4 |

These data show that consistent compositions are produced from various starting populations.

### EXAMPLE 8

The following data show a time course of the production of IFN-γ, IL-4 and IL-2 (ELISA; pg/ml) as a function of days in culture for various samples from three different cancer subjects using the methods herein. Th1 differentiation correlates with IFN-γ production for each subject.

| | IFN-γ | IL-4 | IL-2 |
|---|---|---|---|
| Subject 1 | | | |
| day 1 | 99.1 | 26.1 | 1029 |
| day 2 | 87.3 | 26.1 | 1651.7 |
| day 3 | 120.3 | 67.5 | 6151.87 |
| day 4 | 174.6 | 58.3 | 1116.8 |
| day 5 | 164.1 | 28.5 | 186.1 |
| day 6 | 187.2 | 26 | 101.2 |
| day 7 | 761.4 | 27.2 | 319 |
| day 8 | 1672.3 | 25 | 50 |
| day 9 | 1521.2 | 25 | 50 |
| day 10 | 2500 | 25 | 50 |
| day 12 | 2500 | 25 | 50 |
| harvest | 1003 | 25 | 150 |
| Subject 2 | | | |
| day 1 | 45 | 15 | 366 |
| day 2 | 60 | 15 | 3000 |
| day 3 | 900 | 78 | 7500 |
| day 4 | 3900 | 108 | 7500 |
| day 5 | 4500 | 15 | 5500 |
| day 6 | 6300 | 15 | 200 |
| day 7 | 6900 | 15 | 3210 |
| day 8 | 6900 | 15 | 783 |
| day 9 | 6900 | 15 | 170 |
| day 10 | 7200 | 15 | 636 |
| day 11 | 7200 | 15 | 1300 |
| day 12 | 7200 | 15 | 1800 |
| harvest | 7200 | 00 | 1585 |
| Subject 3 | | | |
| day 4 | 120.1 | 92.6 | 152.1 |
| day 5 | 154.6 | 129.9 | 159.1 |
| day 6 | 193.8 | 76.9 | 150 |
| day 7 | 290.8 | 28.14 | 150 |
| day 9 | 910.9 | 25 | 150 |
| day 12 | 7387 | 25 | 150 |
| harvest | 7000 | 25 | 150 |

These data also demonstrate that IFN-γ, and thus, Th1 differentiation, peaks between about day 9 to day 12.

Since modifications will be apparent to those of skill in this art, it is intended that this invention be limited only by the scope of the appended claims.

1. A method for producing a highly pure population of polyclonal Th1 memory cells, comprising:
collecting source material from a subject;
purifying T-cells from the source material; and
activating the T-cells a minimum of 3 times at 2-4 day intervals, whereby a highly pure population of polyclonal Th1 memory cells are produced.

2. The method of claim 1, wherein the T-cells are purified CD4+ cells.

3. The method of claim 2, wherein the CD4+ cells are purified by positive selection

4. The method of claim 3 wherein the CD4+ cells are purged of CD45RO+ cells

5. The method of claim 1, wherein the source material is purged of platelets

6. The method of claim 4, wherein the source material is purged of platelets

7. The method of claim 1, wherein the source material is purged of monocytes.

8. The method of claim 6, wherein the source material is purged of monocytes.

9. The method of claim 1, wherein the activation of T-cells is effected by contacting the cells with immobilized anti-CD3 and anti-CD28 mAbs.

10. The method of claim 9 where the anti-CD3 and anti-CD28 mAbs are immobilized on immunomagnetic beads.

11. The method of claim 10, wherein the beads are initially administered to the purified T-cells at a 3:1 bead:cell ratio and subsequently at a 1:1 bead:cell ratio.

12. A method comprising:
(a) collecting a sample of mononuclear cells from a subject with a disease characterized by either an excess of Th2 cytokine activity or lack of Th1 cytokine activity; and
(b) processing the mononuclear cells ex vivo without the use of any exogenous cytokines to produce an expanded population of highly pure Th1 memory cells.

13. The method of claim 12, further comprising:
(c) infusing the Th1 memory cells into a subject, thereby altering the Th1/Th2 cell balance of the subject.

14. The method of claim 13, wherein the subject is the donor.

15. The method of claim 13, wherein the expanded population comprises at least 10⁹ Th1 cells.

16. The method of claim 15, wherein the 10⁹ cells are in a volume of about 1 liter or less.

17. The method of claim 12, wherein the disease is selected from the group consisting of diseases characterized by suppression of the cellular immune response or by over-expression of the humoral immune response.

18. The method of claim 12, wherein the disease is selected from the group consisting of cancer, infectious diseases, autoimmune and allergic diseases.

19. The method of claim 12, wherein processing is effected by a method, comprising: purifying CD3+ cells from the mononuclear cells.

20. The method of claim 12, wherein processing is effected by a method, comprising purifying CD3+ CD4+ cells from the mononuclear cells.

21. The method of claim 12, wherein processing is effected by a method, comprising purifying CD3+, CD4+, CD45RA+ cells from the mononuclear cells.

22. The method of claim 12, wherein processing is effected by a method, comprising:
(i) reducing the platelet concentration in the sample;
(ii) purging the CD45RO+ cells from the population of mononuclear cells;
(iii) purifying by positive selection a population of CD4+, CD45RA+ cells;
(iv) activating the resulting CD4+ cells in the absence of exogenous cytokines with immobilized anti-CD3/anti-CD28 mAb;
(v) periodically restimulating with immobilized anti-CD3/anti-CD28 mAb.

23. The method of claim 22, wherein the cells are restimulated every 2-3 days with immobilized anti-CD3/anti-CD28 mAb for a total of 10-14 days.

24. The method of claim 23, further comprising:
(c) infusing the Th1 memory cells into a subject, thereby altering the Th1/Th2 cell balance of the subject.

25. The method of claim 24, wherein the subject is the donor.

26. The method of claim 24, wherein the expanded population comprises at least 10⁹ Th1 cells.

27. The method of claim 26, wherein the 10⁹ cells are in a volume of about 1 liter or less.

28. The method of claim 12, wherein processing is effected by a method, comprising:
(i) reducing the number of platelets in the sample;
(ii) purging macrophages from the sample;
(iii) purging the CD45RO+ cells from the sample
(iv) purifying by positive selection a population of CD4+, CD45RA+ cells;
(v) activating the CD4+ cells in the absence of exogenous cytokines with immobilized anti-CD3/anti-CD28 mAb; and
(vi) periodically restimulating with immobilized anti-CD3/anti-CD28 mAb.

29. The method of claim 28, wherein the cells are restimulated every 2-3 days with immobilized anti-CD3/anti-CD28 mAb for a total of 10-14 days.

30. The method of claim 28, further comprising:
(c) infusing the Th1 memory cells into a subject, thereby altering the Th1/Th2 cell balance of the subject.

31. The method of claim 28, wherein the subject is the donor.

32. The method of claim 28, wherein the expanded population comprises at least 10⁹ Th1 cells.

33. The method of claim 30, wherein the 10 cells are in a volume of about 1 liter or less.

34. A composition comprising at least 70% polyclonal memory Th1 cells.

35. The composition of claim 34, comprising at least 10 Th1 memory cells.

36. The composition of claim 35 that has density of cells greater than about 10⁶ cells per ml.

37. The method of claim 1, wherein the polyclonal Th1 memory cells are activated.

38. The composition of claim 34, wherein the Th1 cells are CD3+, CD4+, CD45RO+, CD62L−, CD44+ and CD25+.

39. A method of treating a disease, comprising:
infusing a composition of claim 34 into a subject with symptoms of a disease, wherein:
the disease is characterized by suppression of the cellular immune response, by over-expression of the humoral immune response, excess Th2 activity or a lack or decreased Th1 activity.

40. The method of claim 39, wherein the disease is selected from the group consisting of cancer, infectious diseases and allergic diseases.

41. A process for producing compositions comprising at least 70% Th1 cells, comprising:
(a) collecting a sample of mononuclear cells from a subject with a disease characterized by either an excess of Th2 cytokine activity or lack of Th1 cytokine activity;
(b) removing platelets from the sample;
(c) removing macrophages from the sample;
(c) depleting CD45RO+ cells from the sample by negative selection;
(d) selecting the CD4+ cells by positive selection; and
(e) expanding and differentiating the selected CD4+ cells by repeatedly stimulating the selected CD4+ cells with immobilized anti-CD3/anti-CD28 antibodies.

42. The method of claim 12, wherein the polyclonal Th1 memory cells are activated.

43. The method of claim 12, wherein the disease is selected from the group consisting of diseases characterized by excess Th2 activity or a lack or decreased Th1 activity.

44. The method of claim 1, wherein the T-cells are activated 3 to 5 times at 2-4 day intervals.

45. The method of claim 1, wherein the source material comprises mononuclear cells.

46. The method of claim 1, wherein the subject is a human cancer patient.

47. A process for producing compositions have an enhanced population of activated polyclonal Th1 memory cells, comprising:
(a) collecting a sample of mononuclear cells from a subject;
(b) expanding and differentiating the mononuclear cells by repeatedly activating T-cells in the mononuclear cell sample in the absence of exogenous growth or differentiation factors, thereby producing a highly pure population of activated polyclonal Th1 memory cells.

48. The method of claim 47, wherein prior to expanding an differentiating the T-cells are purified from the mononuclear cells.

49. The method of claim 48 where the T-cells purified from the mononuclear cells are selected from the group consisting of CD3+ cells, CD4+ cells, CD4+, CD45RA+ cells and CD4+, CD45RO+ cells.

50. A method for expanding T-cells from cancer patients without the use of exogenous cytokines, comprising:
(a) collecting a mononuclear cell sample from a cancer patient;
(b) purging platelets from the mononuclear cells; and
(c) activating the cells with immobilized anti-CD3/anti-CD28 mAbs, wherein all steps are performed in the absence of exogenous cytokines.

51. A composition of cells, comprising at least about 10⁹ cells, wherein at least about 70% of the cells are polyclonal Th1 memory cells.

52. The composition of claim 51, wherein the Th1 cells are activated.

53. The composition of claim 51 that is in a volume of about a liter or less.

54. A composition of polyclonal Th1 memory cells produced by the method of claim 1.

55. A composition of activated polyclonal Th1 memory cells produced by the method of claim 47.

56. A combination, comprising a composition of claim 34 and an immunizing antigen.
